(19) United States
(12) Patent Application Publication
(10) Pub. No.: US 2014/0303494 A1
(43) Pub. Date: Oct. 9, 2014

(54) NEEDLE GUIDANCE SYSTEM

(71) Applicant: Lumoptik LLC, Shaker Heights, OH (US)

(72) Inventors: Thomas I. Janicki, Shaker Heights, OH (US); Arthur Anthony Janicki, Cincinnati, OH (US); Oscar Thomas Janicki, North Olmsted, OH (US)

(73) Assignee: LUMOPTIK LLC, Shaker Heights, OH (US)

(21) Appl. No.: 14/351,286

(22) PCT Filed: Oct. 15, 2012
(86) PCT No.: PCT/US12/60278

§ 371 Date: Apr. 11, 2014

Related U.S. Application Data
(60) Provisional application No. 61/548,929, filed on Oct. 19, 2011.

(51) Int. Cl. A61B 5/06; A61B 5/00

(52) U.S. Cl. CPC A61B 5/061; A61B 5/0059
USPC 600/424

(57) ABSTRACT

A system and device to determine the location of a needle as it penetrates through a tissue and into a desired site, such as the epidural space, are described. The system (1000) contains a light guide (1020), a light source (1040), a light sensor (1050), and a logic unit (1060). When the tip of the needle (1010) traverses the relatively dense Ligamantum Flavum, the reflecting plane of the ligament is positioned at or near zero distance relative to the tip of the device. Once the tip enters a less dense epidural space the distance to the reflecting plane becomes greater than zero thus producing a drop in intensity of the reflected light. That drop in the intensity of the reflected light is measured by the light sensor and interpreted by a logic unit to be consistent with entry into the epidural space, allowing the system to provide a signal indicating entry into the epidural space.

### CROSS-REFERENCE TO RELATED APPLICATION

This application claims priority to U.S. Provisional application No. 61/546,929, entitled “Needle Guidance System”, filed Oct. 13, 2011, the disclosure of which is incorporated by reference.

### FIELD OF THE INVENTION

The present application is directed to the field of needle guidance systems, particularly optical guidance systems.

### BACKGROUND OF THE INVENTION

In the epidural use, the insertion of needles into the epidural space is currently a blind procedure requiring secondary indirect confirmation using a loss of resistance method, or direct confirmation by injection of dye and confirmatory X-ray. Insertion of the tip of an epidural needle into the epidural space without perforation of the dural sac requires significant expertise and training. An “epidural space” is a potential space measuring 2-8 mm in depth (distance between ligamentum flavum and dural sac). If the epidural needle is not advanced sufficiently past ligamentum flavum, the epidural space is not reached. Alternatively, if the tip of the needle is advanced too far, the dural sac may be punctured resulting in leakage of spinal fluid. If a puncture is recognized, typically anesthesia is converted from epidural anesthesia to spinal anesthesia. If the puncture goes unrecognized, severe complications arising from overdose or excessive anesthetic solution in the subdural space may result. The composition and color of ligamentum flavum can assist in its identification as via optical spectroscopy and single wave length light sources. However, those methods are cumbersome and require specialized equipment.

U.S. Publication No. 2009/0099501 to Chang et al., discloses a device for providing needle localization information to a medical professional in real time. Chang's device requires at least two different wave lengths of light, preferably from a laser, and uses data related to their absorbance and reflection to discriminate the tissue types, as the needle travels through different layers of tissue. However, Chang's device is expensive, particularly due to the need for single wavelength light source and dual sensors.

There is a need for an improved, more cost effective needle guidance system.

It is an object of the invention to provide an improved guidance system for guiding a medical instrument or device in a patient, such as a needle, particularly an epidural needle.

It is a further object of the invention to provide an improved method for guiding a needle in a patient to a desired site.

### SUMMARY OF THE INVENTION

The needle guidance system, device, kits, and methods described herein provide direct and objective real-time confirmation of entry of a needle or other device into a desired site in a patient's body, and are particularly useful for confirmation of entry of a needle into the epidural space. The needle guidance system, when used to guide an epidural needle, may replace or supplement loss of resistance syringes and injection of dye and confirmatory x-rays in epidural steroid injections (pain management procedure). This may enable other health providers to engage in the use of epidural steroid and other injections for treatment of neck, back, upper and lower extremity pain, increasing access to care.

The system contains a light guide, a light source, a light sensor, and a logic unit. When the tip of the needle traverses the relatively dense Ligamantum Flavum, the reflecting plane of the ligament is positioned at or near zero distance relative to the tip of the device. Once the tip enters a less dense epidural space the distance to the reflecting plane becomes greater than zero thus producing a drop in intensity of the reflected light. That drop in the intensity of the reflected light is measured by the light sensor and interpreted by a logic unit to be consistent with entry into the epidural space, allowing the system to provide a signal indicating entry into the epidural space.

The system incorporates a penetration sensor which uses LABA (Light Assisted Breach Assessment) Technology, such as disclosed in U.S. application Ser. No. 13/352,109, filed Jan. 17, 2012. The disclosure of which is incorporated herein by reference. The penetration sensor detects differences in densities of penetrated layers and structures. In one example, uncollimated light is transmitted through the light guide placed in the center of the needle. When the tip of the needle traverses the relatively dense Ligamantum Flavum, the reflecting plane of the ligament is positioned at zero or near zero distance relative to the tip of the light guide. Once the tip of the needle, and hence the light guide, enters a less dense epidural space the distance to the reflecting plane becomes greater than zero thus producing a drop in intensity of the reflected light. That drop in the intensity of the reflected light is measured by the light sensor and interpreted by a logic unit to be consistent with entry into the epidural space, allowing the logic unit to provide a visual, tactile and/or acoustic signal, indicating entry into the epidural space.

### BRIEF DESCRIPTION OF THE DRAWINGS

FIG. 1 is a functional diagram of a needle guidance system, where the distal portion of the light guide is in optical communication with a y-split light guide, where one end of the y-split light guide is in optical communication with a light source, and the other end is in optical communication with a light sensor.

FIG. 2 is a partial cut-away, cross-sectional view of a needle tip and a light guide, where the light guide is an optical fiber pair.

FIG. 3 is a partial cut-away, cross-sectional view of the distal portion of a needle and the distal portion of a light guide, where the light guide is an annular optical path.

FIG. 4A is a partially cut-away view of an epidural needle guidance system; FIG. 4B is a magnified, cross-sectional view of the distal tip of the needle and light guide.

FIG. 5 is an exploded view of the epidural needle guidance system illustrated in FIG. 4A.

FIG. 6 is a functional block diagram of an embodiment of a logic unit for a needle guidance system.

FIG. 7 is a side view of the epidural needle and a light transmissive stylet, which fits inside the lumen of the needle, where the stylet can serve as a light guide for the needle guidance system.

FIG. 8A is a partially exploded cross-sectional view of an epidural needle guidance system which contains a light transmissive stylet as the light guide. FIG. 8B is a magnified, cross-sectional view of the distal tip of the needle and light guide. FIG. 8C is a perspective view of the handpiece for the needle guidance system, which preferably includes a pull tab to activate the device. FIG. 8D is a perspective partial view of the proximal end of the stylet, which attaches to the distal end of the handpiece to assemble the needle guidance system.

FIG. 9 is a block diagram of the electrical system for an exemplary epidural needle guidance system, which contains both a pressure sensor and a light sensor. As shown in FIG. 9, the system may also contain means for wireless communication with a remote device, such as a Bluetooth module.

### DETAILED DESCRIPTION OF THE INVENTION

A system, device, kit, and method are described herein to assist an operator of a device, typically a medical professional in determining the location of a needle as it penetrates through a tissue and into a desired site, such as positioning an epidural needle within the epidural space.

### I. Needle Guidance System

The system contains a light guide, a light source, a light sensor, and a logic unit. The light guide has a diameter, shape and length suitable for insertion into and removal from the lumen of a needle, preferably an epidural needle. The needle has a path for fluid communication therethrough between a distal sharp end and a proximal end. The path is generally referred to as the lumen or bore of the needle. Optionally, the system includes a pressure sensor for measuring the change in pressure in the distal end of the lumen of the needle.

The devices described herein contain a needle with the needle guidance system attached thereto, such that the light guide is in the lumen of the needle, preferably with the distal end of the light guide at or near the distal end of the needle.

Preferably the system contains a handle. In some preferred embodiments, the logic unit is housed in the handle. Optionally, the system also contains a locking connector which connects the needle to the system, optionally via the light guide and/or handle.

The logic unit interprets the data provided by the light sensor, and optionally the pressure sensor, to determine if the distal end of the needle has entered a less dense area, such as the epidural space. Then the logic unit triggers one or more indicators which produce a signal, such as a visual, tactile or acoustic signal, to indicate to the operator that the needle has entered into the epidural space. The signal may be provided on the needle guidance device, such as on a user interface on the device. Alternatively, the data may be transmitted to a remote site, such as a separate monitor. The data may be transmitted wirelessly, such as via Bluetooth, or another transmission system.

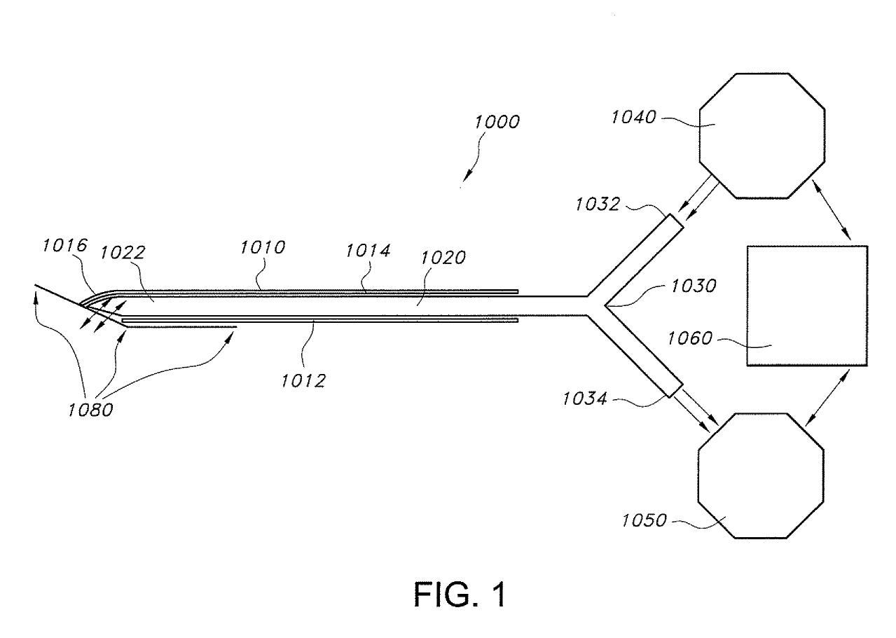

An exemplary epidural needle guidance system 1000 inserted into an epidural needle 1010 is illustrated in FIG. 1. The needle contains needle walls 1012 which define a central lumen 1014. A light guide 1020 of the needle guidance system fits inside the central lumen and its distal end 1022 aligns with the distal tip 1016 of the needle. In some embodiments, when fully inserted in the needle, the distal end 1022 of the light guide 1020 is at the distal tip 1016 of the epidural needle 1010, such that the distal end of the light guide is flush with the distal tip of the needle. In other embodiments, the distal tip of the light guide is inside the lumen of the needle, in an area proximal to the distal tip of the needle. In this embodiment, the light guide typically contains a lens at its distal tip, such that the outer surface of the lens is flush with the distal tip of the needle.

The light guide directs light from the light source 1040 to the distal end 1016 of the needle. Preferably, as illustrated in FIG. 1, the light guide is in optical communication with a Y split 1030 light guide. One end 1032 of the Y split 1030 terminates at and/or is in optical communication with a light source 1040, and the second end 1034 terminates at and/or is in optical communication with a light sensor 1050. As more completely discussed below, both the light source 1040 and the light sensor 1050 may be controlled by and/or connected to a logic unit 1060.

Preferably the needle guidance system contains a continuous positive air or fluid pressure within the lumen 1014 of the needle. The positive pressure inside the lumen of the needle pushes the light reflecting plane away from the distal tip of the needle, which further decreases that intensity of the reflected light when the needle enters the epidural space. This increases the difference in the sensor's measurements between the intensity of reflected light in the ligamentum flavum and the intensity of reflected light in the epidural space. The positive pressure may be provided by any suitable device, including but not limited to a medical pressure bulb, an air pump, or self-refilling limited capacity air bladder, and the like.

Additionally, the needle guidance system preferably contains a pressure sensor, which provides data to the logic unity regarding the pressure in the lumen of the needle. When the distal tip enters the epidural space, the pressure in the lumen drops. The pressure data is transmitted from the pressure sensor to the logic unit. The pressure data can be analyzed by the logic unit and provided as an output to the operator (or other medical professional). The output can be in any suitable form, such as a visual, audible or tactile signal. In one embodiment, the pressure data is visually displayed on user interface, such as in the form of a graph. The inclusion of a pressure sensor in the system can increase robustness of analysis to determine entry of the distal tip of the needle into the epidural space.

A. Light Guide

A variety of light guides may be included in the system and device described herein. In one embodiment, such as illustrated in FIG. 1, the light guide 1020 may be an optical fiber in optical communication with a Y split 1030 light guide. The light guide may comprise a single optical fiber, an optical fiber pair (or plurality), an annular optical path, a light transmissive plastic support insert such as a stylet, or a combination thereof.

As used herein, the term “optical” when used to describe a material that can be used as a light guide refers to a material that is capable of transmitting light to the distal end of the light guide with sufficient energy for reflected light to be detected by the light sensor. Suitable materials include optical quality materials, and also less perfect, but still optically transmissive materials, such as optically transmissive plastics and similar inert materials.

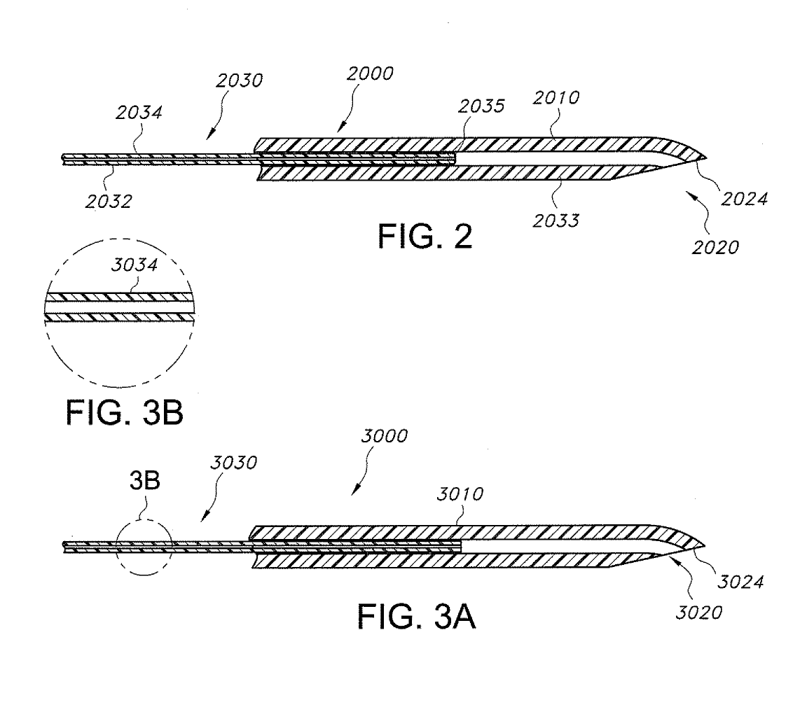

FIG. 2 illustrates a portion of an exemplary device containing an epidural needle guidance system (EGS) in the lumen of a needle in which the light guide is an optical fiber pair 2032, 2034. The light guide has a suitable size and shape to fit inside the lumen 2020 of the needle 2010. In the embodiment of an optical fiber pair 2032, 2034, a proximal end (not shown in FIG. 2) of a transmitting fiber, 2032, may be in optical communication with a light source (not shown in FIG. 2), and a proximal end (not shown in FIG. 2) of receiving fiber, 2034, may be configured for optical communication with a light sensor for detecting reflected light (not shown in FIG. 2). Distal ends of the fiber pair 2032, 2034 may be advanced through the lumen 2020 to substantially align with the distal end 2024 of the needle or the tissue plane.

FIG. 3 illustrates a portion of an exemplary device containing an epidural needle guidance system (EGS) in the lumen of a needle in which the light guide is an annular optical path. The light guide 3030 is sized to fit inside the bore 3020 of the needle. The light guide 3030 may comprise an annular optical path 3034 configured to provide an optical path from light sending and light sensing logic at the proximal end (not shown in FIG. 3) to the distal end 3024 as well as a path for fluid communication or catheter access. For example, a proximal end of the light guide may be in optical communication with a splitter (not shown in FIG. 3), in further optical communication with a light source (not shown in FIG. 3) on one hand and a light detector (not shown in FIG. 3) on the other. The light guide 3030 may be advanced through the bore 3020 to substantially align with the tissue plane at the distal end 3024.

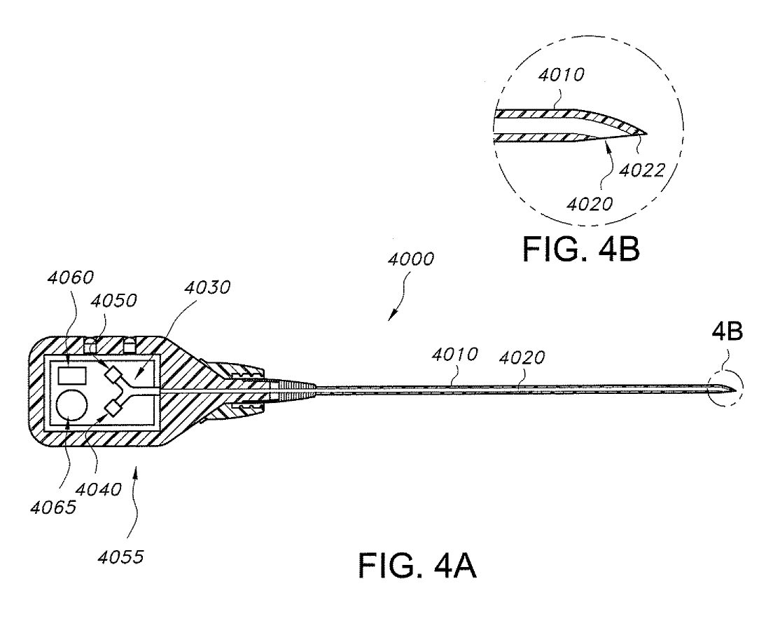

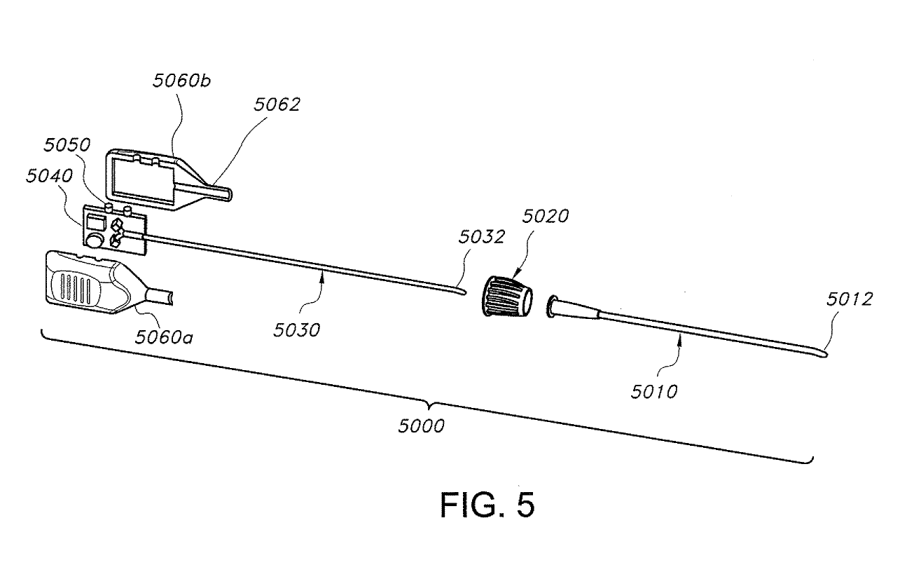

FIGS. 4 and 5 illustrate an exemplary device containing an epidural needle guidance system (EGS) in the lumen of a needle, which contains a single optical fiber as the light guide.

With respect to FIG. 4, in one embodiment a device 4000 includes an epidural needle 4010 having a removable light guide 4020 within the bore of the needle. At the distal end, shown enlarged in FIG. 4B, the light guide 4020 may be appropriately beveled (such as angled at a 45° angle or other suitable angle) in its distal aspect as part of the piercing instrument. The proximal end of the light guide 4020 is divided into two light guides, for example via a y-splitter 4030, although effective splitting may be achieved with a dichroic mirror, beamsplitter, or the like. One end of splitter 4030 terminates at light source 4040 such as a light emitting diode and the other end terminates at a light sensor 4050.

Both the light source 4040 and light sensor 4050 may be disposed in a handle 4055 and connected with logic 4060, a power supply 4065 and circuitry (not shown) to operably inter-connect some or all of the components. For example, logic 4060 may control the light source 4040 to an illuminating condition. Logic 4060 may further detect reflected light intensity levels from light sensor 4050, and optionally detect pressure changes from a pressure sensor (not shown), and provide one or more visual, auditory or other output signals.

With reference to FIG. 5, the device 5000 may contain a conventional epidural needle 5010 connected to a needle guidance system with a locking connector 5020, such as a Luer lock. The light guide 5030 may be inserted into the bore of needle 5010 such that the distal ends are aligned. The light guide 5030 connects on the proximal side to a logic unit 5040 that may include an indicator, such as an LED 5050, to signal unit readiness, penetration or other desirable alerts. Logic unit 5040 may be configured to fit within a handle 5060. The distal end 5062 of the handle may be configured to accept the locking connector 5020. The handle may also be configured to show the LED 5050 and protect the logic unit 5040. In one example, the indicators include at least two different colored LEDs, such as a red LED indicator and a green LED indicator.

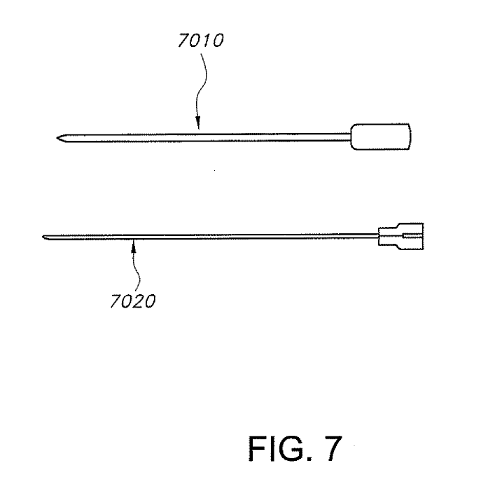

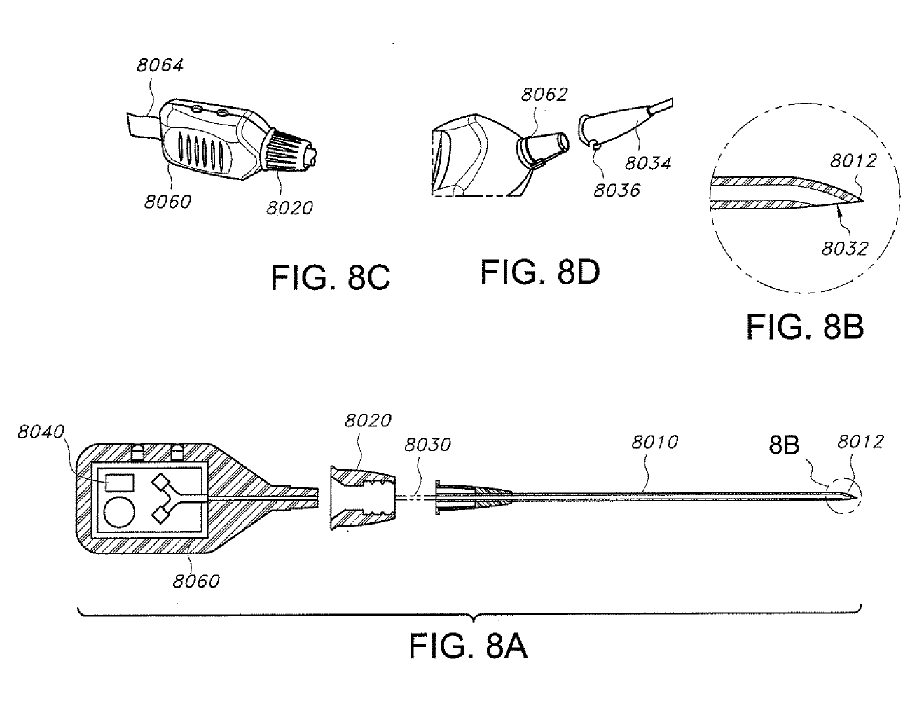

FIGS. 7 and 8 illustrate an exemplary device containing an epidural needle guidance system (EGS) in the lumen of a needle, where the light guide is an optically transmissive stylet. With reference to FIGS. 7 and 8, in one embodiment, an optically transmissive stylet, such as a Tuohy Needle Plastic Stylet 7020, 8020, may be used as the light guide and the device may be configured to establish an optical path from the logic unit 8040 in the handle 8060 to a proximal end of the stylet held in place with the needle 8010 by connector 8020. The distal end of the handle is configured to connect with and attach to, such as with a notch or snap-fit, and align the proximal end of the stylet 8034 in the desired orientation. The connector 8020 is configured to fit over the distal end of the stylet and attach it to the proximal end 8062 of the handle. In a preferred embodiment, the handle is configured to reduce or prevent slippage when the operator's hand grips the device. For example, the handle may contain a texture (e.g. ribbed or raised surfaces) on opposite sides of the handle. Preferably the handle contains a removable tab configured to activate the device after the needle guidance system is connected to and assembled with the needle.

### 1. Angle of the Distal Tip of the Light Guide

The distal tip of the light guide is preferably cut at a 90° angle, a 45° angle, or any other desirable angle. The distal tip of the light guide, preferably an optical fiber, may be bare or contain a lens, depending on the need. A lens may be added to a light guide to increase light transmission and capture of reflected light by the light guide.

### 2. Lens

The lens on the distal tip of the light guide may be formed from any suitable material that transmits light, and preferably preserves the evanescent wave at the distal tip of the light guide.

In one embodiment a lens is formed by cutting an optical fiber at a 90° angle, such that the distal tip of the light guide is inside the lumen of the needle, and is short of (and proximal to) the distal tip of the needle, forming a void and then filling the void with a sterile, transparent, biologically inert fluid, such as saline, to form a lens. The fluid fills the beveled portion of the needle, such that the fiber itself does not have direct contact with the reflective plane.

Alternatively, acrylic or another transparent material which improves light transmission and reflection, and preferably also preserves the evanescent wave, can be directly applied to the distal tip of a fiber to form a lens. This is particularly useful for an optical fiber cut at a 45° angle or another angle less than 90° or greater than 90°.

B. Light Source

The light source may be any non-collimated light, such as a light emitting diode (LED), incandescent light, and the like. The light source provides un-collimated light of any desired wavelength, or multiple wavelengths. In general, the light is preferably a non-collimated light from LED or other non-collimated light producing device. Such a non-collimated source is less expensive to implement than a collimated light source, such as a laser. However, in some embodiments, coherent light sources may be used.

Preferably the light source is a compact electrically driven light source emitting adequate radiant flux to allow measurements by the light sensor. One embodiment is a low-power white-light broadband visible spectrum LED with a molded plastic lens. However the light source may be chosen to enhance measurement of particular penetration tissues. Possible light source optical parameters include narrow or broadband spectral content from the UV to infrared region, linear or circular polarization, coherent or incoherent light, and intensity pulsing or modulation. These characteristics are readily available with off-the-shelf light sources, such as LEDs, laser diodes, incandescent bulbs, and discharge lamps, combined with the use of optional wavelength converting phosphors and optical filters.

The light source may contain a lens for efficient optical coupling between the light source and the optical fiber. Light pulsing may be used to reduce power consumption, and light modulation may reduce ambient light interference. Preferably, the light provided by the light source is modulated at a suitable frequency. This allows for elimination of background, ambient light and increases the dynamic range of the useful signal. Suitable frequencies include, but are not limited to, 1 kHz, 2 kHz, and the like.

In one embodiment, in order to eliminate the impact of ambient light on the measurement of reflected light intensity, two measurements are performed during each measurement cycle. First, the return light is measured with the LED off (i. e., only ambient light is measured). Then the returned light is measured with the LED on (i e., a sum of ambient light and the true return light is measured). Subsequently the results are subtracted, such as by the logic unit, one from another yielding the intensity value of a true return light.

The measurements may be performed continuously. By way of example, light measurements may be taken every 500 μs, i.e., true return light measurements are obtained every 1 ms (1 kHz frequency). The results are accumulated, and preferably the result is averaged at regular intervals (e.g. every 50 ms or other suitable interval). Averaging is implemented to reduce the level of noise in the measurement.

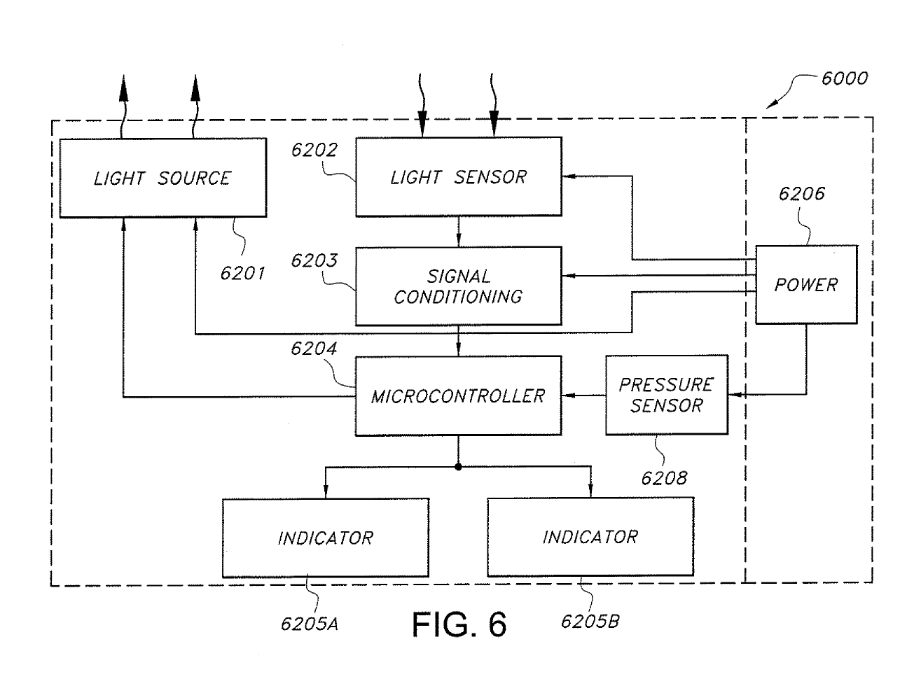

As shown in FIG. 6, the system contains a light source 6201 and a light sensor 6202. Optionally, the system also contains a pressure sensor 6208. The light source, light sensor, and optional pressure sensor, are each powered by one or more power sources, preferably one power source 6206 or the microcontroller 6204. The light source 6201 may be pulsed or modulated by a digital or analog control signal from microcontroller 6204, and the light source 6201 may receive power from the microcontroller 6204 control signal or power source 6206.

C. Light Sensor

With reference to FIGS. 1 and 6, the light sensor 1050, 6202 is configured to receive reflected light from the light guide, such as a stylet or optical fiber, and convert the reflected light to an electrical signal. The light sensor may be spectrally matched to the light source 1040, 6201 and may be sensitive to optical wavelengths of interest. The response time is selected to be adequate to detect light intensity variations during penetration of different tissues or spaces in a patient's body by the distal end of the needle to determine when the distal tip reaches the desired site.

An example of a suitable light sensor is a photodiode with a molded plastic lens. Other suitable light sensors may be selected based on cost, sensitivity, and response time. Alternative suitable light sensors include a light dependent resistor, photovoltaic cell, phototransistor, CCD, microbolometer, photomultiplier tube, or other electro-optical sensor matched to the light source.

Optionally, optical filters may be applied to the light sensor to restrict the measurement spectrum or polarization, to reduce interference, or increase measurement sensitivity. The light sensor may contain a lens for efficient optical coupling between the optical fiber and light sensor.

The light sensor may use power from the logic unit or a power source.

D. Logic Unit

As generally used herein, “logic” refers to hardware, software, firmware, or combinations thereof that perform a function or an action, and/or cause a function or an action from another component. For example, depending on the application or needs of the system or device, logic may include a software controlled microprocessor, discrete logic such as an application specific integrated circuit (ASIC), a programmed logic device, memory device containing instructions, or the like. Logic may also be fully embodied as software configured to perform the desired action or function.

As generally used herein “software” refers to one or more computer readable and/or executable instructions that cause a computer or other electronic device to perform functions, actions, and/or behave in a desired manner. The instructions may be embodied in various forms such as routines, algorithms, modules, or programs including separated applications or code from dynamically linked libraries. Software may also be implemented in various forms such as a stand-alone program, a function call, a servlet, an applet, instructions stored in a memory, part of an operating system or other type of executable instructions. As is appreciated by one of ordinary skill in the art, the form of software is dependent on, for example, requirements of a desired application, the environment it runs on, and/or the desires of a designer/programmer or the like.

With reference to FIG. 6, a logic unit assembly 6000 includes control logic to analyze the data provided by the sensor(s), preferably a light sensor 6202 and a pressure sensor 6208, and provide outputs regarding system status information, when the appropriate conditions are reached. Preferably the logic unit assembly also includes signal conditioning circuitry 6203 prior to and in electrical communication with the light sensor 6202, and optionally a pressure sensor 6208, and the control logic 6204, to convert the light sensor output to a suitable input for the control logic. Preferably, the value of pressure can be provided directly from the pressure sensor to the logic unity, and there is no need to perform further processing of pressure result.

Preferably the logic unit assembly also includes one or more indicators 6205 to communicate the level of penetration of the distal tip of the needle to the operator. Preferably the system also includes an indicator 6205 that displays the system state to the operator.

### a. Control Logic

Control logic may include a single-chip microcontroller 6204 with integrated program memory, RAM, timers, and an analog-to-digital converter that implements logic to control the system. The microcontroller 6204 may sample the signal-conditioned or raw signal from the light sensor 6202, and/or pressure sensor 6208, execute an algorithm to analyze signal from the light sensor 6202, and/or pressure sensor 6208, preferably from both, and output system status information to one or more indicator 6205. The microcontroller 6204 may send a digital or analog control signal to light source 6201 to modulate or pulse the light intensity. In the case of an analog control signal, the microcontroller 6204 contains a digital to analog converter. The microcontroller 6204 receives power from power source 6206 and may control power functions including power saving mode and energy storage (e.g. battery charging). The microcontroller 6204 may be highly integrated to include some or all of the logical assembly components, or may use external components for individual features.

With reference to FIG. 1, the logic unit 1060 analyzes the light sensor data for changes in the intensity of the reflected light. Preferably the logic unit also analyzes that pressure sensor data for changes in pressure in the lumen of the needle. Both the intensity of the reflected light and the pressure in the lumen of the needle change, depending on the tissues or spaces encountered by the distal tip of the needle. For example, upon reaching the epidural space a measurable reduction in reflected light may be sensed by the light sensor and a decrease in the pressure in the needle may be sensed by the pressure sensor. Upon a threshold level of change in intensity of reflected light, optionally in combination with a drop in the pressure in the lumen of the needle, the logic unit produces a user feedback indicator in any suitable form, such as a visual, acoustic, or kinetic signal, or a combination thereof.

### b. Signal Conditioning Circuitry

Signal conditioning circuitry converts the light sensor electrical output to a suitable voltage range for sampling by the control logic, such as in analog-to-digital converter circuitry. The signal conditioning circuitry 6203 may include passive or active circuitry. Additionally, frequency selective filtering may be applied to reduce unwanted noise and perform anti-aliasing before analog-to-digital conversion.

The preferred embodiment for signal conditioning 6203 of a photodiode light sensor 6202 is a transimpedance amplifier circuit with a low-pass filter characteristic for anti-aliasing. Other light sensors 6202 have well known application circuits that may be implemented with low cost. System performance may allow a simple and lowest cost resistor-capacitor (RC) signal conditioning circuitry 6203.

Signal conditioning circuitry can be used, if necessary, to convert the pressure sensor output to a suitable voltage range. However, in the preferred embodiment, such conversion is not necessary.

The signal conditioning circuitry may use power from power source 6206.

### c. Indicator

The system contains one or more signal generators, also referred to herein as indicators, to produce one or more signals. The signal(s) communicate to the operator one or more of the conditions, including but not limited to:
(1) state of the system (e.g. Power On/Off), (2) Penetration of the distal tip of the device, (3) Pressure and/or dynamic change in pressure over time in the lumen, (4) intensity and/or dynamic change in the intensity of reflected light at the distal tip, and/or (5) communication ability (e.g. wireless communication enabled).

The indicator 6205 displays the system state to the operator. System states may include indication of power on/off, system ready, and level of penetration of the distal end of the system. The indicator provides a signal, such as a visual, tactile or acoustic signal.

In one embodiment, the system includes two indicator LEDs 6205a and 6205b to visually alert the operator. One 6205a of the LEDs may indicate system power or readiness, and the other 6205b may indicate level of penetration.

Alternatively, or additionally, the indicator 6205 may provide an audible signal such as a beep, tone, speech, or some other audible signaling method, or a visual signal 6205 that varies intensity, color, shape, text, or symbols to indicate the system state. Further, alternatively or additionally, the indicator may provide a tactile signal, such as vibration or a variety of vibrational patterns.

For example, the indicator may include one or more LED indicators, such as one or more colored lights; an audible sound; or tactile signal, such as vibration or different vibrational patterns; or a combination thereof.

### d. Power Source

The power source 6206 for the logical assembly 6200 may include an internal battery or an external power source 6206. Preferably the assembly 6200 uses an internal battery, although power may be provided through an external power jack that overrides internal battery power, on-board energy storage in the form of a rechargeable or non-rechargeable battery or other energy storage device, with control of energy management may be performed by microcontroller 6204. The power may be supplied to components using electrically conductive wires, wireless power transfer using inductive, RF, or optical power transfer, or other methods.

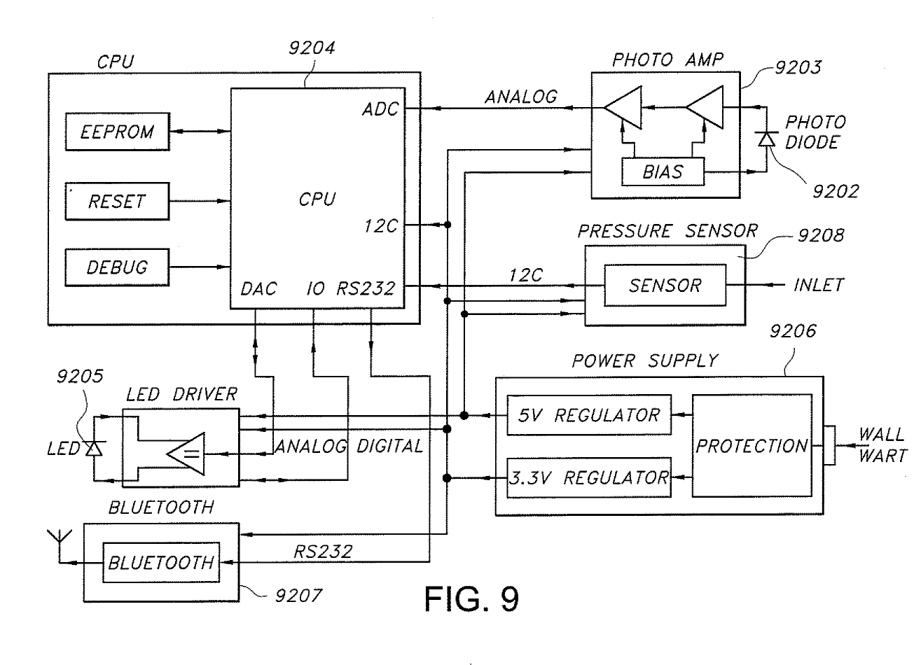

FIG. 9 provides an exemplary block diagram of electrical components that can be used in the needle guidance system. The system contains a microprocessor, such as a digital signal processor (DSP) processor 9204. The input data for the processor comes from a signal conditioning unit, such as a photo amplifier 9203, which receives the light signal from the light sensor 9202, and from a pressure sensor 9208, which measures pressure in the lumen of the needle. The microprocessor 9204 sends output data to an indicator, such as the LED driver produces a light signal, and to a wireless communication module 9207, such as a Bluetooth module, which transmits data to an external computer.

The system is powered from a power module 9206, such as a wall wart generating DC voltage, which can be converted into two voltages to power the subsystems, for example, 5V and 3.3V.

### II. Method of Using the Needle Guidance System

The Epidural Needle Guidance System 1000 uses differences in density of ligamentum flavum and epidural space and the behavior of those structures around the distal tip of the epidural needle during its insertion in the patient to determine the location of the distal tip of the needle.

The position of the tip of the epidural needle may be determined or confirmed from the change in the intensity of the reflected light from the reflecting plane. Optionally, the position of the tip may be determined from the change in pressure associated with passing the distal tip of the needle from one reflecting plane into another. Preferably, both measurements of changes in intensity of reflected light and changes in pressure in the distal end of the needle are used to determine the position of the distal tip of the needle.

A general method for determining the position of the distal tip of the needle can be understood with reference to the figures, particularly FIG. 1 and FIG. 6. The light receiving interface is the distal tip 1022 of the light guide 1020 within the epidural needle. The pressure sensor 6208 measures the pressure of air or fluid in distal end of the lumen of the needle.

The system operates in zero or near zero distance from the reflecting plane.

In use, as illustrated in FIGS. 1 and 6, the light source 1040, 6201 is powered by a suitable power source 6206, and light is carried through the Y split light guide 1030 or other suitable light guide, down the length of the light guide 1020 to a tissue plane 1080. Certain amounts of the light are reflected off the tissue plane 1080 and travel back along the light guide 1020, through the Y split 1030 and are detected by the light sensor 1050, 6202. The intensity of the reflected light is then measured and interpreted by the logic unit 1060, 6204. Optionally, the electrical output from the light sensor and/or pressure sensor is converted to a suitable voltage range by signal conditioning circuitry 6203 prior to analysis by the microcontroller 6204. The intensity of the reflected light changes depending on the tissues or spaces encountered. Upon a threshold level of change in reflected light, and optionally pressure change in the needle, the indicator(s) 6205 produce one or more signals.

In use the system detects intensity of the reflected light returned by a penetrated barrier and pressure changes in the needle. The reflecting plane is at zero or near zero distance from the distal tip of the light guide when the distal tip of the needle penetrates a dense tissue, such as the ligamentum flavum. At the moment of piercing of the outer shell of the tissue and progression into a less dense environment, the distance from the distal tip of the light guide to the reflecting plane becomes greater than zero. Any change in near zero distance environments produces much greater changes in intensity than those measured from reflections in the 1 mm or 2 mm range. According to light intensity formula (I=1/r²) the intensity of the reflected light is inversely proportional to the square of the distance. Indeed, reflections in the zero or near zero distance environments from non-collimated light approach reflection percentages of collimated light due to the reduced opportunity of light to scatter in the zero or near zero distance environments.

When used in the epidural environment, inserting an epidural needle 1010 through ligamentum flavum toward the epidural space, the high density of the ligament being perforated produces its tight apposition against the tip of the needle, thus distance between distal tip of the light guide and tissue approaches zero mm during penetration. Upon entering the epidural space, a structure of much lower density, the distance between the light interface within the epidural needle and the reflecting plane of epidural space becomes non-zero, that is, greater than zero mm. The reflected light conveyed by the light guide 1020, through the Y split 1030 to the light sensor 1050 will decrease dramatically upon entering the epidural space causing the logic unit 1060 to detect the decreased reflection. The logic unit 1060 may then provide the operator with an appropriate signal, such as a warning or notice. The signal can be in any suitable manner to indicate that the distal end of the needle is entering the epidural space, including visual, tactile or acoustic signals.

Different light guides, such as illustrated in FIGS. 2 and 3 may be used in place of the y-split light guide. Regardless of light guide implementation, the needle and light guide assembly may be advanced to an appropriate depth or tissue penetration as assisted by the light sending and sensing logic. Once the distal end of the needle is placed in the desired location in the patient's body, the optical components may be removed as needed and appropriate medical care provided through the lumen in the needle or fluid path.

Similar mechanism of action will occur in other non-epidural and non-medical applications. The difference in the densities of the perforated layers will result in measurement of differences of the intensities of the reflected light during penetration of each of the layers. Such data may then be processed by a logical unit to create appropriate output.

The needle guidance system may be a disposable device for use in medical diagnostic and therapeutic arenas such as hospitals, surgery centers, doctors' offices, invasive radiology and others. The system may be operated by medical professionals, such as nurses, lab technicians, or physicians.

In typical use, the anesthetist prepares the patient for the insertion of the epidural needle as usual. The needle guidance system is removed from the sterile package and either fitted into the well of the transparent plastic stylet, or the usual stylet is removed from the epidural needle and a replacement light transmissive stylet (e.g. a transparent plastic stylet, such as illustrated in FIG. 7) or light guide with attached handle and/or logical components, such as illustrated in FIGS. 1, 2, 3, 4, and 5, is inserted into the standard epidural needle. Preferably, an activation tab (see, e.g. FIG. 8C) is pulled out of the device activating the electronic circuit. In a preferred embodiment, when the electronic circuit is activated an indicator provides a signal to the user, such as by illuminating a light, e.g., a LED light.

After inserting the light guide into the lumen of the needle and attaching the needle guidance system to the epidural needle, the distal tip of the epidural needle is then slowly advanced by the medical professional, typically an anesthetist, through the skin, subcutaneous fat and ligamentum flavum toward the epidural space.

In one preferred embodiment, a signal, e.g. LED light, stays on indicating proper function of the device and then a second signal is provided when entry into the epidural space is detected by the system, such as due to a decrease in the intensity of reflected light, optionally in combination with a pressure drop. The second signal can be any suitable signal, such as a second, LED, or other warning or notice signal, e.g. tactile or audible.

The anesthetist or other medical professional then disengages the needle guidance system and removes the stylet or light guide from the epidural needle, if necessary. The anesthetist may then proceed conventionally with the needle in the epidural space. The components of the needle guidance system may then be disposed.

Continuing with the explanation of use in the epidural environment, the system provides anesthetists, or pain specialists with immediate and objective confirmation of entry into the epidural space, preventing him/her from advancing the needle too far and producing undesired entry into the subdural space (wet tap) with its potential for all associated complications.

Additionally, the needle guidance system may be used by other health providers, allowing them to engage in the use of epidural steroid and other injections for treatment of neck, back, upper and lower extremity pain, and thereby increasing access to care.

Following proper insertion in a patient, the needle may be used to provide epidural anesthesia; epidural steroid injections, such as for treatment of back pain. Alternatively the needle guidance system may be used to insert a device, such as electrical stimulator leads, into the epidural space.

In other embodiments, the needle guidance system described herein may be configured to be suitable to guide orthopedic, neurosurgical or surgical piercing instruments. In yet a further embodiment, the guidance system may be configured to be used in non-medical piercing and/or perforating equipment.

### 3. Kits

The needle guidance system may be a sterile, self-contained disposable device, which includes a light guide of a length and diameter suitable to fit within the central lumens of standard epidural and spinal needles. Preferably the light guide is a y-split light guide. In another embodiment, the light guide may be retracted, cut or otherwise modified to length. In still other embodiments, fitting a suitable light guide may have a suitable diameter and length to fit within orthopedic, neurosurgical and/or surgical piercing instruments, or within non-medical piercing or perforating equipment.

The needle guidance system may be provided in sterile packaging. In one embodiment the kit contains a single needle guidance system with the system preassembled (i.e. light guide connected to the handle) and ready for insertion into and assembly to a needle. The needle guidance system may contain any suitable light guide, as described above. In some embodiments, the kit contains an optically transmissive stylet as the light guide. In other embodiments the kit contains an optical fiber as the light guide.

In another embodiment the kit contains a plurality of light guides, optionally of different lengths and diameters, to allow a user to select the appropriate light guide for the device to be guided. After selecting the appropriate light guide, the user attaches it at its proximal end to the handle such that it is in optical communication with the light sensor and light source and places the light guide through the needle, and finally attaches the needle to the needle guidance system.

Preferably the kit contains instructions to guide the user in proper assembly, use and optionally disposal, of the needle guidance system. a single light guide, and either fitted into the well of the transparent plastic stylet, or the usual stylet is removed from the epidural needle and a replacement light transmissive stylet (e.g. a transparent plastic stylet, such as illustrated in FIG. 7) or light guide with attached handle and/or logical components, such as illustrated in FIGS. 1, 2, 3, 4, and 5, is inserted into the standard epidural needle.

### EXAMPLE

### Example

### Swine Study Testing Various Wavelengths of Light

Two separate sessions of testing on pig models were conducted to test the needle guidance system. The needle guidance system used in these tests contained a Y split optical fiber, as the light guide, fitted and secured within a Tuohy epidural needle in a fashion that guaranteed that the distal end (cut at a 45° angle) of the fiber was flush with the distal end of the epidural needle. The light source was a white LED light source. ZOOM II Optical Power Meter from Optical Wavelength Laboratories was used as the light sensor to measure intensity of reflected light in μW.

The same anesthetist preformed each of the insertions of the epidural needles. The anesthetist had over 10 years of clinical experience in insertions of epidural needles for epidural anesthesia.

Three anesthetized pigs were tested at the Case Western Reserve University animal facility on two separate occasions.

Initially the anesthetist used a loss of resistance method on one of the pigs for insertion of the epidural needle to determine best technique of insertion of the needle in the pig model. Afterwards, in the tests conducted on two of the pigs, the anesthetist proceeded with insertion of the epidural needle, which contained the needle guidance system described above.

Measurements of the intensity of the reflected light were taken at different wavelengths at the point of “presumed” ligamantum flavum; then the needle was advanced further until a significant drop in the intensity of the reflected light was observed on the ZOOM II Optical Power Meter screen using one of the wavelengths. Then the forward advance of the needle was then stopped and a number of measurements of the intensity of the reflected light were taken at different wavelengths.

Following completion of the measurements of the intensity of reflected light, the optical fiber was removed from the lumen of the needle, and 1 cc-1.5 cc of oil paint was injected through the needle into the area surrounding the distal tip of the needle.

This procedure was repeated on a number of levels in two separate pigs. Following the completion of the testing, the pigs were euthanized and the epidural area was surgically exposed to confirm location of the injected oil paint.

Table 1 provides the percent change in the intensity measurements in two different fibers at a variety of different wavelengths (850 nm, 1300 nm, 1300 nm, 1310 nm, 1490 nm, and 1550 nm). The values listed beneath the second and third columns in the table represent percentage of decrease of the intensity of reflected light upon reaching epidural space as compared to intensity of reflected light within ligamentum flavum.

TABLE 1
Change in intensity measured in Two different fibers in
different lighting conditions at different wavelengths

| | % Decrease in | % Decrease in |
|---|---|---|
| | Intensity | Intensity |
| Wavelength | Fiber #1 | Fiber #2 |
| 850 nm | 17% | 44% |
| 1300 nm | 23% | 45% |
| 1310 nm | 23% | 41% |
| 1490 nm | 19% | 40% |
| 1550 nm | 19% | 43% |

Analysis of the injected oil paint in the euthanized pigs confirmed successful identification of epidural space. Additionally, the data relating to the decrease in intensity of reflected light reveals a predictable drop in the intensity of the reflected light with similar relative drop at all wavelengths of light measured. Fact that the drop occurs regardless of wavelength indicates that the intensity of the reflected light at zero and near zero distance environment is more useful than color (i.e. wavelength) dependent identification to identify the location of the distal tip of the needle.

I claim:

1. A medical penetration detection system for insertion into the lumen of a needle comprising:
a light guide having a suitable width and length for fitting inside the lumen of the needle, wherein the proximal end of the light guide is in selective optical communication with a light sensor and a light source; wherein the light source is a non-collimated light source; and
a logic unit configured to sense a decrease in an amount of reflected light from the distal end of the light guide, wherein in use the decrease corresponds to a desired needle penetration level.

2. The medical penetration detection system of claim 1, wherein the light guide comprises a plurality of discrete fiber optic paths and at least a proximal side of one of the plurality of discrete fiber optic paths is in optical communication with the non-collimated light source and at least a proximal side of another of the plurality of discrete fiber optic paths is in optical communication with the light sensor.

3. The medical penetration detection system of claim 1, wherein the light guide comprises an optically transmissive stylet, comprising a proximal side in optical communication with a splitter, wherein the splitter is in temporally controlled optical communication with the light source and the light sensor.

4. The medical penetration detection system of claim 1, wherein the system further comprises a splitter, and wherein the light guide comprises an optical fiber, wherein a proximal side of the light guide is in optical communication with the splitter, and wherein the splitter is in temporally controlled optical communication with the light source and the light sensor.

5. The medical penetration detection system of any one of claims 1 to 4, further comprising a pressure sensor configured to measure the pressure in the bore of the needle.

6. The medical penetration detection system of any one of claims 1 to 5, wherein the light guide comprises a lens at its distal tip.

7. The medical penetration detection system of any one of claims 1 to 6, wherein the logic unit comprises a microprocessor.

8. The medical penetration detection system of claim 7, wherein the microprocessor is programmed to receive and analyze data from the light sensor and optionally from the pressure sensor.

9. The medical penetration detection system of claim 8, further comprising one or more indicators configured to provide one or more signals, wherein the indicators are in electrical communication with the microprocessor, and wherein one of the indicators is configured to provide a signal to alert an operator when the microprocessor determines that there is a decrease in the amount of reflected light.

10. The medical penetration detection system of any one of claims 1 to 9, further comprising a handle, wherein the logic unit is housed inside the handle, and wherein the handle is configured to attach to the proximal end of the needle.

11. A device comprising a needle for insertion in a patient and the medical penetration detection system of any one of the claims 1 to 10.

12. The device of claim 11, wherein the needle is an epidural needle.

13. A kit comprising the medical penetration detection system of any one of claims 1 to 10 in sterile packaging.

14. The kit of claims 13, comprising a plurality of light guides.

15. A method for determining the location of a needle as it penetrates a patient's body comprising inserting the distal tip of the needle of the device of claim 11 into the patient's body until an indicator provides a signal, wherein the signal corresponds with a decrease amount of reflected light received from the distal end of the needle.

16. The method of claim 15, wherein the decrease in the amount of reflected light corresponds with when the distal tip of the needle enters the epidural space in the patient.

17. The method of any one of claim 15 or 16, further comprising removing the medical penetration detection system of any one of claims 1 to 10 from the needle after the indicator provides the signal.

18. The method of claims 17, further comprising delivering anesthesia or other therapeutics to a patient through the lumen of the needle after removal of the medical penetration detection system.
